(19) United States
(12) Patent Application Publication
(10) Pub. No.: US 2016/0287735 A1
(43) Pub. Date: Oct. 6, 2016

(54) PLASMA INSTALLATION WITH A SEPARATELY TRANSPORTABLE VESSEL

(71) Applicant: Christof-Herbert Diener, Nagold (DE)

(72) Inventor: Christof-Herbert Diener, Nagold (DE)

(21) Appl. No.: 15/184,844

(22) Filed: Jun. 16, 2016

Related U.S. Application Data
(63) Continuation of application No. PCT/EP2014/077029, filed on Dec. 9, 2014.

(30) Foreign Application Priority Data
Dec. 20, 2013 (DE) 10 2013 226 814.9

(51) Int. Cl. A61L 2/00; A61L 2/14

(52) U.S. Cl. CPC A61L 2/0011; A61L 2/14; A61L 2202/21

(57) ABSTRACT

A plasma installation includes at least two separate components being a vessel for an object to be processed and a plasma unit having an electrical plasma source for igniting a plasma in the vessel. The plasma unit has no additional subassembly for other media so that the plasma unit can be constructed in a structurally particularly simple manner and readily operated. In a particularly preferred manner, the vessel comprises a vessel body of glass. The vessel body is preferably constructed in one piece and surrounds the object completely. The plasma can be ignited spaced apart from the plasma unit by means of a high-frequency generator, an induction coil and/or a magnetron of the electrical plasma source.

### CROSS-REFERENCE TO RELATED APPLICATIONS

This continuation application claims priority to PCT/EP2014/077029 filed on Dec. 9, 2014 which has published as WO 2015/091104 A1 and also the German application number 10 2013 226 814.9 filed on Dec. 20, 2013, the entire contents of which are fully incorporated herein with these references.

### DESCRIPTION

1. Field of the Invention

The invention relates to a plasma installation for processing an object, in particular an implant, wherein the plasma installation has a closable vessel for receiving the object and a plasma unit having an electrical plasma source for producing a plasma in the vessel, wherein the vessel is constructed as a unit which can be transported independently of the plasma unit, wherein the plasma can be produced in the vessel directly by the electrical plasma source of the plasma unit and the vessel has a vessel body which is constructed substantially from a dielectric material.

2. Background of the Invention

It is known to process an object in a plasma. It is further known to process implants, in particular tooth implants, in a plasma shortly before implantation. Such a plasma processing method is known, for example, from DE 195 02 133 A1.

The killing even of multi-resistant germs in a plasma is described by Peter Trechow in the VDI news of 22 Nov. 2013, Edition 47. In this instance, it is set out that, as a result of plasma-based surface functionalization of implants, it is possible to selectively make them particularly attractive to cells and consequently to accelerate an ingrowth of permanent implants. The use of a plasma on implants consequently enables cell-attractive properties to be combined with antimicrobial properties.

The cleaning of implants in a plasma is further known from the Article “Microscopical and microbiologic characterization of customized titanium abutments after different cleaning procedures”, Clinical Oral Implants Research, 0, 2012/1-9.

US 2013/0230426 A1 discloses a plasma installation for sterilization of implants in a vacuum container. The vacuum container is connected to a pump unit in order to produce a reduced pressure in the container during the plasma processing operation. Such a device for plasma sterilization is further known from WO 2010/044669 A1.

The plasma processing of the implant is intended to be carried out directly before the implantation in order to achieve the best possible effect. To this end, it has previously been necessary to install complex and costly plasma installations in doctors' surgeries or hospitals. These plasma installations must generally be connected to a pump and a plurality of gas bottles, whereby the complexity and costs for the plasma installation increase. Furthermore, such plasma installations have to be operated by specially trained personnel.

In contrast, it has become known from DE 10 2004 049 783 B4 and WO 03/059400 A1 to divide a plasma installation into a plasma unit with an electrical plasma source and a vessel which can be transported independently of the plasma unit.

However, the known vessels have to be provided in a complex manner with electrodes. These electrodes have to be contacted for the plasma processing of the object. The construction of the vessels is thereby cost-intensive. Furthermore, the contacting of the vessels has to be carried out by trained personnel.

WO 2011/116984 A2 discloses a plasma installation with a vessel, wherein the vessel has no electrodes for igniting a plasma. In order to ignite the plasma, however, the vessel has to be brought into direct contact with an electrode structure of the plasma installation.

U.S. Pat. No. 6,558,621 B1 discloses enclosing an implant in a vessel directly after a plasma processing operation and discharging it from the plasma processing chamber.

It is further known from WO 97/44503 A1 to sterilize the inner side of a container in a plasma chamber by means of a plasma processing operation.

An object of the present invention in this regard is to provide a plasma installation by means of which objects, in particular implants, can be plasma-processed in a simple and cost-effective manner.

### SUMMARY OF THE INVENTION

This object is achieved according to the invention with a plasma installation having the features of the independent claim. The dependent patent claims set out preferred developments of such a plasma installation.

The plasma installation for processing an object, in particular an implant, comprises a closable vessel for receiving the object and a plasma unit having an electrical plasma source for producing a plasma in the vessel, wherein the vessel is constructed as a unit which can be transported independently of the plasma unit, and the plasma can be produced directly by the electrical plasma source of the plasma unit, wherein the vessel has a vessel body which is constructed substantially from a dielectric material, wherein the plasma unit does not have a gas connection and/or a pump and is constructed in such a manner that the plasma can be ignited when the vessel is located with spacing from the plasma unit.

The plasma unit consequently comprises an independently transportable, closable vessel. The object, in particular the implant, can be received in the vessel. The manufacturer of the object can therefore close and deliver the object in the vessel. The expression “independently transportable vessel” is intended in this instance to be understood to be a vessel which is not permanently connected to the plasma unit. In particular the vessel is not permanently connected to the plasma unit by means of hoses for gases and/or electrical lines.

Directly before the object is used, the vessel can be brought directly to the place of use close to the plasma unit. The plasma unit then produces a plasma in the vessel. The plasma processes the object in the vessel. Subsequently, the vessel can be opened, the object removed from the vessel and used (in particular implanted). The plasma unit must thereby have only one electrical subassembly for producing the plasma, that is to say, the electrical plasma source. The plasma unit can thereby be constructed in a particularly simple and cost-effective manner.

The electrical plasma source produces the plasma directly in the vessel. This direct production of the plasma is intended in this instance to be understood to be a “remote action” of the electrical plasma source on the vessel. The vessel can thereby be constructed so as to be free from electrodes. The costs for producing the vessel are thereby significantly reduced, particularly since electrodes for igniting the plasma have to be constructed with a precisely defined spacing in the vessel. The plasma unit is in this instance constructed in such a manner that the plasma can be ignited when the vessel is located with spacing from the plasma unit. The vessel consequently only has to be moved close to the plasma unit in order to ignite a plasma in the vessel. A contacting of the vessel by trained personnel can thereby be dispensed with.

In a particularly preferred manner, the vessel can be permanently closed in an air-tight manner.

The vessel may be provided with a rapid-fit coupling, for example, of Cajon or Swagelok, in order to enable rapid and simple pressure adjustment in the vessel.

In a particularly preferred manner, the vessel is provided with an irreversible temperature indicator in order to be able to verify whether the object has already been plasma-processed in the vessel. The temperature indicator is in this instance preferably constructed for use in a temperature range between 30° C. and 100° C., preferably between 40° C. and 60° C. As a result of such a temperature indicator, it can further be observed whether the necessary duration of the plasma processing operation has already been reached.

The plasma unit does not have a gas connection and/or a pump. The plasma unit is thereby constructed in a particularly simple manner. Furthermore, a plasma unit can be operated in a particularly simple manner without media connections.

The plasma installation is constructed in such a manner that the plasma unit, in particular during the plasma processing, has no connection to the vessel. That is to say, there is neither an electrical nor a fluid line between the vessel and plasma unit. Preferably, there is also no fixed mechanical connection between the plasma unit and vessel. The plasma unit preferably does not have any closed plasma chamber.

In a particularly preferred manner, the plasma unit has a housing with a recess for receiving the vessel, wherein the housing only partially surrounds the vessel.

The vessel body may be constructed from ceramic material or a resistant plastics material. In a particularly preferred manner, the vessel body is constructed completely from a dielectric material.

In a particularly preferred embodiment of the invention, the vessel body is at least partially, in particular substantially, preferably completely, constructed from glass. Glass is transparent so that a user of the plasma installation can observe the ignition of the plasma in the vessel, and is further particularly plasma-resistant.

The vessel may be constructed from a metal pipe having a glass pane.

The vessel body is in this instance preferably constructed in the form of an ampoule. Such ampoules are particularly suitable for processing an implant since ampoules are already known and widely used in doctors' surgeries and clinics. The procedure with ampoules is therefore already commonplace to the staff in clinics and doctors' surgeries. The ampoule may have at least one hermetically closed tip.

The vessel may have a stopper for closing the vessel body. Vessel bodies and stoppers in this instance preferably each have a ground portion in order to be able to close the vessel body with the stopper in an air-tight manner.

In another preferred embodiment of the invention, the vessel cannot be opened without being destroyed after receiving the object. Such a vessel may, on the one hand, be produced in a particularly cost-effective manner and has, on the other hand, the advantage of a particularly high level of sealing. Furthermore, with such a vessel, it can immediately be identified whether it is still in the state “originally packed” by the manufacturer or has already been opened or manipulated. The vessel body may in this instance be constructed in an integral manner and surround the object completely.

In a particularly preferred manner, a vessel body of the vessel is in this instance constructed in the form of a snap-off ampoule. A snap-off ampoule is intended to be understood to be a glass ampoule which has to be broken for opening. The snap-off ampoule may have a/an, in particular annular, desired breaking location. The desired breaking location may, for example, be scratched in the vessel body. Alternatively or additionally, a desired breaking location may be constructed in the form of a burnt-in enamel ring.

The vessel body is, for simple production of the vessel body and for easy introduction into the plasma unit, preferably constructed in a substantially rotationally symmetrical manner with respect to the longitudinal axis thereof. In a particularly preferred manner, the vessel body has in this instance a cylindrical portion.

The vessel body is preferably integral and in particular constructed in such a manner that it completely surrounds an object which is received therein.

In order to be able to ignite a low-pressure plasma in the vessel body, the gas pressure in the vessel body may be less than 1 bar, in particular less than 0.1 bar, preferably less than 0.01 bar, in a particularly preferred manner less than 0.001 bar.

The plasma installation according to the invention may be used to etch a surface of the object in the vessel. In this instance, the vessel preferably contains an etching gas, for example, tetrafluoromethane (CF₄), hexafluoroethane (C₂F₆), perfluoropropane (C₃F₈), octafluorocyclobutane (C₄F₈), sulphur hexafluoride (SF₆), and/or nitrogen trifluoride (NF₃). The vessel preferably contains a fluorine-containing gas in order to etch a titanium surface of the object in the vessel.

The plasma processing method according to the invention can further be used to activate a surface of the object. To this end, a gas in the form of argon (Ar), oxygen (O₂) and/or air is preferably contained in the vessel. Alternatively or additionally, water (H₂O), in particular in the gaseous state, may be contained in the vessel.

The vessel may further contain an amine-containing gas, in particular methylamine (CH₅N), dimethylamine (C₂H₇N), trimethylamine (C₃H₉N), ammonia (NH₃), betaine (C₅H₁₁NO₂) and/or butylamine (C₄H₁₁N). The amine-containing gas is in a particularly preferred manner used in the case of an object in the form of an implant since it has been shown that biological cells grow particularly well on implants which have been plasma-processed with an amine-containing gas.

The filling of the vessel is greatly simplified when atmospheric pressure, that is to say, ambient pressure, is intended to be present in the vessel after closure. In order to enable an ignition of the plasma in spite of the atmospheric pressure, the vessel is in this instance filled to a level greater than 40%, in particular to a level greater than 50%, preferably to a level greater than 60%, in a particularly preferred manner to a level greater than 70% with helium (He).

The plasma processing method according to the invention can further be used to apply a layer to the object in the vessel. To this end, the vessel may contain a compound which decomposes in plasma, for example, a silane compound.

The electrical plasma source of the plasma unit may have for direct plasma production in the vessel a high-frequency generator and electrodes which are electrically connected to the high-frequency generator and between which the vessel can be at least partially introduced. The electrodes are in this instance preferably constructed in a substantially annular manner in order to be able to introduce the vessel in a simple manner between the electrodes. In a particularly preferred manner, the electrodes are constructed around the recess of the housing of the plasma unit, which recess is constructed to receive the vessel.

In order to directly produce the plasma in the vessel, the electrical plasma source may have a magnetron, wherein the microwave radiation of the magnetron can pass at least partially through the vessel. In order to be able to direct the electromagnetic radiation of the magnetron in a selective manner onto the vessel, the magnetron may be connected to an antenna for emission of the electromagnetic radiation. In a particularly preferred manner, the antenna is constructed around the recess of the housing of the plasma unit, which recess is constructed to receive the vessel.

The direct production of the plasma in the vessel can further be achieved by the electrical plasma source having an induction coil in which the vessel can be at least partially introduced. In a particularly preferred manner, the induction coil is constructed around the recess of the housing of the plasma unit, which recess is constructed to receive the vessel.

The invention further relates to a method for processing an object, in particular an implant, according to the patent claim having the method steps of:

a) placing the object in a vessel, which is substantially constructed from a dielectric material;

b) adjusting the gas pressure and/or the gas composition in the vessel;

c) closing the vessel;

d) transporting the vessel to a plasma unit;

e) directly igniting a plasma in the vessel by means of an electrical plasma source of the plasma unit, wherein the plasma unit does not have a gas connection and/or a pump and the vessel is spaced apart from the plasma unit.

As set out above, this method according to the invention affords the particular advantage of being able to use a structurally very simple plasma unit at the premises of a user of the object, for example, at the premises of a user of an implant in a clinic or a doctor's surgery. The object, in particular the implant, can be delivered from the manufacturer of the object in the vessel to the user of the object. In the case of an implant, the plasma processing can take place shortly before the operation, that is to say, a maximum of approximately 1 hour before the operation. The object, or the implant, can then be used with a freshly coated, etched and/or activated surface. The vessel is at a distance, that is to say, spaced apart, from the plasma unit when the plasma is ignited. The vessel thereby has to be moved only approximately into the vicinity of the plasma unit. A precise positioning is not necessary.

The plasma installation used preferably has one or more of the features described above.

In a particularly preferred manner, a vessel body of glass is used.

Preferably, there is used a vessel body which is constructed in an integral manner and which in particular surrounds the object completely.

In a particularly preferred embodiment of the invention, there is used a vessel which cannot be opened without being destroyed after receiving the object.

In the method step b), a gas pressure of less than 1 bar, in particular less than 0.1 bar, preferably less than 0.01 bar, in a particularly preferred manner less than 0.001 bar, is preferably adjusted.

In order to ignite the plasma, it is possible to use in the plasma unit an electrical plasma source which has a high-frequency generator and electrodes which are electrically connected to the high-frequency generator and between which the vessel is at least partially introduced.

In order to directly ignite the plasma, there may further be used an electrical plasma source which has a magnetron, wherein the microwave radiation of the magnetron passes at least partially through the vessel.

In a particularly preferred manner, the method according to the invention is used to activate an implant, in particular a tooth implant, shortly before implantation. An adhesion of the implant can thereby be carried out avoiding surface etchings, in particular avoiding the use of hydrofluoric acid (HF).

Preferably, the parameters for carrying out the method according to the invention are selected in such a manner that the surface energy of the object is at least doubled. Tests with objects of titanium have shown that the surface energy of 43 mN/m can be increased to far above 100 mN/m when the method according to the invention is carried out. A particularly efficient activation of the surface of the object is thereby achieved.

In a particularly preferred manner, the method according to the invention is used to carry out a plasma processing operation in order to improve growth of an implant. In this instance, the important aspect is not sterilizing the implant in the plasma, but instead activating it. The implant is consequently already sterile in the vessel prior to the plasma processing.

Finally, the invention according to one of the patent claims relates to the use of a vessel which is constructed without any electrodes and which can be transported independently of a plasma unit and which at least partially comprises glass, for plasma processing of an object, in particular an implant, in a plasma unit, wherein the plasma unit does not have a gas connection and/or a pump.

The vessel used preferably has one or more of the features described above.

In a particularly preferred manner, the vessel is used in a doctor's surgery, in particular a dental surgery or a clinic.

A particularly suitable use of the vessel involves activating an implant, in particular a tooth implant, shortly before the processing operation so that it can be readily used without further chemical processing, in particular avoiding surface etchings and the like.

Other features and advantages of the invention will be appreciated from the following detailed description of a plurality of embodiments of the invention, with reference to the figures of the drawings which show details significant to the invention, and from the patent claims.

The features shown in the drawings are illustrated in such a manner that the individual features according to the invention can be made clearly visible. The different features can be implemented individually per se or together in any combination in variants of the invention.

### BRIEF DESCRIPTION OF THE DRAWINGS

In the drawings:

FIG. 1 is a schematic view of a first plasma installation;

FIG. 2 is a schematic view of a second plasma installation;

FIG. 3 is a schematic view of a third plasma installation;

FIG. 4a shows a plasma installation according to U.S. Pat. No. 6,558,621 B1;

FIG. 4b shows a plasma installation according to the invention in comparison with the plasma installation according to FIG. 4a,

FIG. 5a is a schematic pressure/time graph of a method carried out according to U.S. Pat. No. 6,558,621 B1; and

FIG. 5b is a schematic pressure/time graph of a method according to the invention compared with the method according to FIG. 5a.

### DETAILED DESCRIPTION OF THE PREFERRED EMBODIMENTS

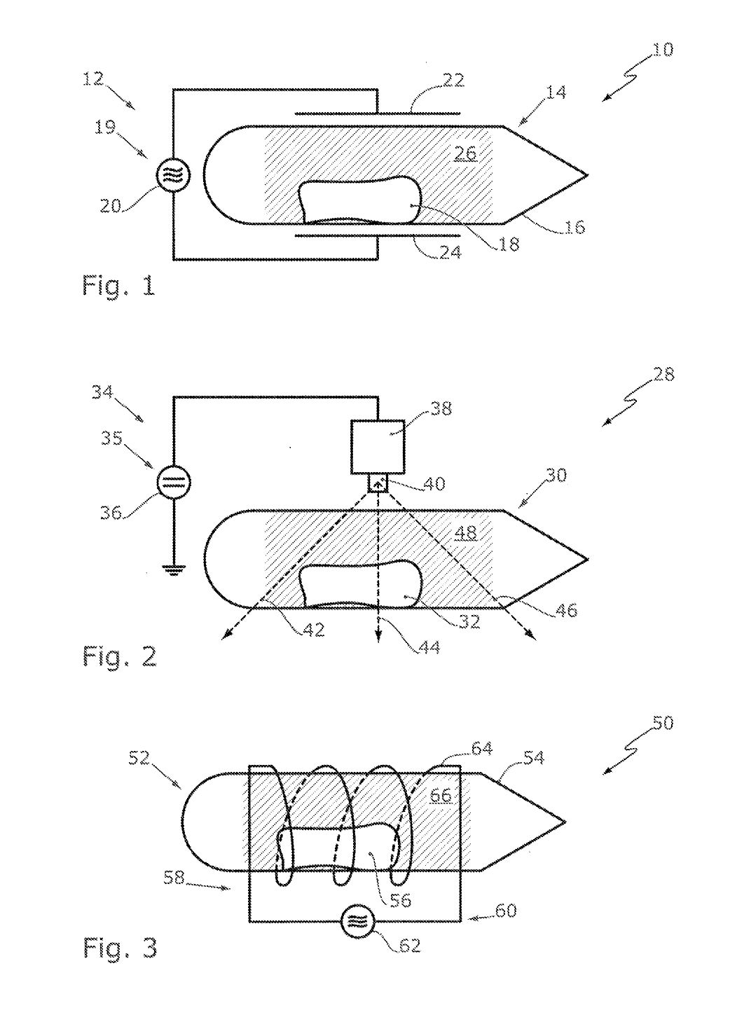

FIG. 1 shows a first plasma installation 10. The first plasma installation 10 has a plasma unit 12 and a vessel 14. The vessel 14 is constructed as a unit which does not have any electrodes and which can be transported independently of the plasma unit 12. The vessel 14 comprises a vessel body 16 of glass. The vessel body 16 encloses an object 18. The object 18 is in the form of a tooth implant.

The object 18 was introduced into the vessel 14 after the production thereof. Subsequently, reduced pressure was produced in the vessel 14 and the vessel 14 was closed in an air-tight manner. The vessel body 16, after receiving the object, that is to say, in the state shown in FIG. 1, cannot be opened without being destroyed. To this end, the vessel body 16 is constructed in the form of a glass ampoule. The vessel 14 can thereby be delivered from the manufacturer of the object 18 to the user, for example, to a dental practice.

The user moves the vessel 14 close to the plasma unit 12. The plasma unit 12 comprises an electrical plasma source 19 which in this instance has a high-frequency generator 20. The high-frequency generator 20 is electrically connected to electrodes 22, 24 of the electrical plasma source 19. By switching on the electrical plasma source 19, a plasma 26 is ignited in the vessel 14 in the region of the electrodes 22, 24. The object 18 is plasma-processed by the plasma 26. In this instance, the vessel 14 is filled with inert gas and oxygen so that the surface of the object 18 is freed from germs and activated.

After the plasma processing, the user can break the vessel body 16 and remove and use the object 18.

The plasma unit 12 is constructed in a structurally particularly simple manner, whereby the productions costs as a result of the plasma unit 12 are approximately one order of magnitude lower than the production costs for a “conventional” plasma unit with a processing chamber which can be pumped out. Furthermore, no media, in particular no gases, have to supplied to and/or discharged from the plasma unit 12. The plasma unit 12 according to the invention can thereby also be used in a particularly simple manner by means of operators who have not been specially trained.

FIG. 2 shows a second plasma installation 28. A vessel 30 having an object 32 are constructed in an identical manner to the vessel 14 or the object 18 according to FIG. 1. In contrast to the plasma unit 12 according to FIG. 1, however, the plasma installation 28 according to FIG. 2 has a plasma unit 34 whose electrical plasma source 35 comprises a direct-current voltage generator 36. The direct-current voltage generator 36 is electrically connected to a magnetron 38 of the electrical plasma source 35.

On the magnetron 38 is an antenna 40 for emitting electromagnetic radiation which is indicated in FIG. 2 by arrows 42, 44, 46. The electromagnetic waves or the electromagnetic radiation is preferably in the microwave range. The electromagnetic radiation is in this instance selected in such a manner that a plasma 48 is ignited in the vessel 30. The vessel 30 is constructed as a unit which can be transported independently of the plasma unit 34.

FIG. 3 shows a third plasma installation 50. The third plasma installation 50 has a vessel 52 which is constructed without any electrodes and which comprises a vessel body 54 of glass, wherein an object 56 is introduced in the vessel body 54.

The third plasma installation 50 comprises a plasma unit 58 in addition to the vessel 52. The vessel 52 is constructed as a unit which can be transported independently of the plasma unit 58. The plasma unit 58 has an electrical plasma source 60 which comprises a high-frequency generator 62. The high-frequency generator 62 is electrically connected to an induction coil 64 for igniting a plasma 66.

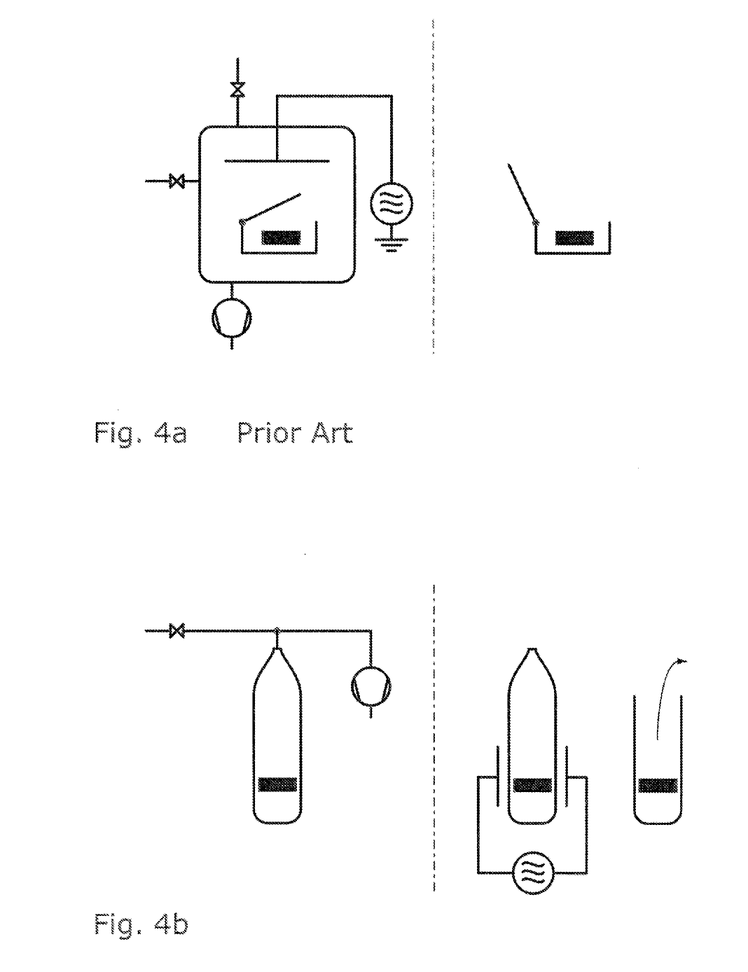

FIG. 4a shows a plasma installation according to U.S. Pat. No. 6,558,621 B1. FIG. 4a is sub-divided into an implant manufacturer side to the left of the broken line and an implant customer side to the right of the broken line. In FIG. 4a, it can be seen that an implant is plasma-processed at the premises of the implant manufacturer with the vessel open. After the plasma processing, the vessel is closed, delivered to the customer and opened at that location before the implantation.

FIG. 4b shows in contrast a plasma installation according to the invention. To the left of the broken line, the implant manufacturer side is illustrated again and to the right of the broken line the implant customer side is illustrated. In FIG. 4b, it can be seen that the implant is introduced into the vessel at the manufacturer's premises and the vessel is provided with a suitable atmosphere and closed. The actual plasma processing is then carried out on the premises of the customer who opens the vessel after the plasma processing and implants the implant. In contrast to the prior art, consequently, a plasma processing of an implant can be carried out directly on the customer's premises, that is to say, in the doctor's surgery or clinic without a plasma installation which is complex to operate having to be provided on the customer's premises.

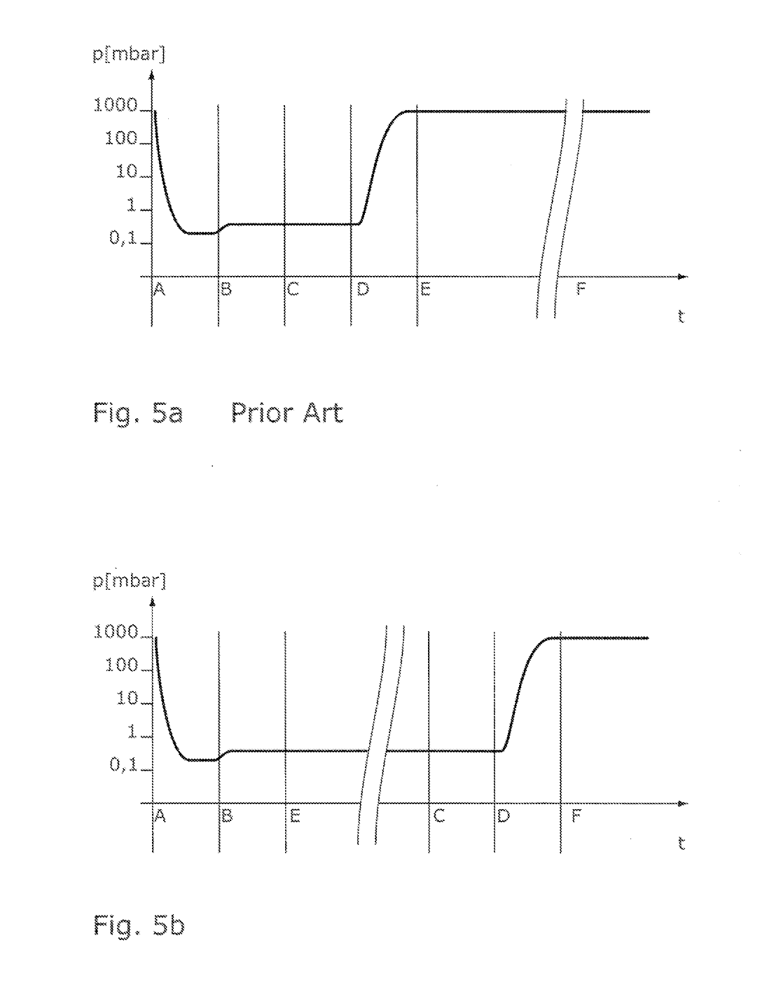

FIG. 5a shows a time (X axis)/pressure (Y axis) graph of a method according to U.S. Pat. No. 6,558,621 B1. According to the prior art, the plasma chamber is first pumped out (A), and there are subsequently carried out a gas stabilization (B), ignition of a plasma (C), ventilation of the plasma chamber (D), packaging/transport/storage (E) and implantation (F). In FIG. 5a, it can be seen that there may be a long period of time between the plasma processing and the implantation when the plasma installation is not constructed directly on the customer's premises.

FIG. 5b shows in contrast a time/pressure graph of a method according to the invention. In this instance, a pumping-out operation (A) is first carried out in the vessel, followed by a gas stabilization (B). The implant is then packaged/transported/stored (E). The plasma processing (C) is carried out directly in the plasma unit on the customer's premises. Since the plasma unit has only electrical connections, it is very simple for the client to operate. Generally, it has only one off and on switch. Directly after the plasma processing, there is carried out the ventilation (D) by opening the vessel and the implantation (F).

As a result of the invention, consequently, the plasma processing can be carried out both at a time directly before the implantation and using a plasma installation which is constructed in the structurally simplest manner possible.

In summary, the invention relates to a plasma installation. The plasma installation comprises at least two separate components, that is to say, a vessel for the object to be processed and a plasma unit with an electrical plasma source for igniting a plasma in the vessel. The plasma unit has no additional subassembly for other media so that the plasma unit can be constructed in a structurally particularly simple manner and can be readily operated. In a particularly preferred manner, the vessel comprises a vessel body of glass. The vessel body is preferably constructed in one piece and surrounds the object completely. The plasma can be ignited in a state spaced apart from the plasma unit by means of a high-frequency generator, an induction coil and/or a magnetron of the electrical plasma source.

What is claimed is:

1. A plasma installation for processing an object in the form of an implant, wherein the plasma installation has a closable vessel for receiving the object and a plasma unit having an electrical plasma source for producing a plasma in the closable vessel, wherein the closable vessel is constructed as a unit which can be transported independently of the plasma unit, wherein the plasma can be produced in the closable vessel directly by the electrical plasma source of the plasma unit and the closable vessel has a vessel body which is constructed substantially from a dielectric material, wherein the plasma unit does not have a gas connection and/or a pump, wherein the plasma unit is constructed in such a manner that the plasma can be ignited when the vessel is located with spacing from the plasma unit, wherein the vessel body is built at least partially from glass and in the form of a snap-off ampoule.

2. The plasma installation according to claim 1, wherein the gas pressure in the vessel is less than 1 bar.

3. The plasma installation according to claim 1, wherein the gas pressure in the vessel substantially corresponds to atmospheric pressure and the vessel is filled to a level greater than 40% with helium.

4. The plasma installation according to claim 1, wherein the electrical plasma source has a high-frequency generator and electrodes which are electrically connected to the high-frequency generator and between which the vessel can be at least partially introduced.

5. The plasma installation according to claim 1, wherein the electrical plasma source has a magnetron and the electromagnetic radiation of the magnetron can pass at least partially through the vessel.

6. The plasma installation according to claim 1, wherein the electrical plasma source has an induction coil in which the vessel can be at least partially introduced.

7. A method for processing an object in the form of an implant, comprising the steps of:
a) placing the object in a vessel having a vessel body, wherein the vessel body is built at least partially from glass and in the form of a snap-off ampoule;
b) adjusting the gas pressure and/or the gas composition in the vessel;
c) closing the vessel;
d) transporting the vessel to a plasma unit;
e) directly igniting a plasma in the vessel by means of an electrical plasma source of the plasma unit, wherein the plasma unit does not have a gas connection and/or a pump and the vessel is spaced apart from the plasma unit.

8. Use of a vessel which is built without any electrodes and which can be transported independently of a plasma unit and which at least partially consists of glass and is constructed in the form of a snap-off ampoule, for plasma processing of an object in a plasma unit, wherein the plasma unit does not have a gas connection or a pump.
